(19) United States
(12) Patent Application Publication
(10) Pub. No.: US 2023/0094298 A1
(43) Pub. Date: Mar. 30, 2023

(54) HIGH YIELD GREEN CALYX HIBISCUS VARIETY YAO GREEN

(71) Applicant: University of the District of Columbia, Washington, DC (US)

(72) Inventors: Matthew Lee Richardson, Silver Spring, MD (US); Yao Afantchao, Washington, DC (US)

(21) Appl. No.: 17/953,971

(22) Filed: Sep. 27, 2022

Related U.S. Application Data
(60) Provisional application No. 63/248,770, filed on Sep. 27, 2021.

(51) Int. Cl. A01H 6/60; A01H 5/02

(52) U.S. Cl. CPC A01H 6/608; A01H 5/02

(57) ABSTRACT

A new variety of edible hibiscus designated ‘Yao green’ characterized by green calyces which is insect resistant and particularly suited for productive grown in the Mid-Atlantic region of the United States.

### BACKGROUND OF THE INVENTION

### Field of the Invention

The present invention relates to varieties of hibiscus plants.

### Description of the Background

As the global population increases, there is an increase in food demand and a decrease in agricultural land. This fact, in conjunction with drastic variations in climatic conditions and natural disasters, has resulted in the investigation of alternative crops that can provide good nutrition, are easily propagated, and tolerate a wide range of production systems and environmental conditions.

Hibiscus sabdariffa L., commonly called red sorrel or roselle, is a hardy shrub native to the tropics of West Africa, is widely grown throughout tropical regions of the world, has high economic value as a food crop, and has a diversity of uses as an ornamental plant and for consumption.

Parts of this plant are often used in traditional medicine as well as in health supplements as they are rich in phytochemicals, particularly flavonols, anthocyanins, polysaccharides, and organic acids. For instance, preparation of herbal drinks, hot and cold beverages, and fermented drinks of the leaves or calyces are traditionally used in Asia and Africa for their diuretic, choleretic, febrifugal, hypoglycemic, and hypotensive effects. The plant is considered traditional medicine in Thailand for kidney and urinary bladder stones. In Bangladesh, the leaves and calyces are eaten as vegetables, and their fiber is used as a jute substitute. Calyces are traded worldwide as an important ingredient for the industrial production of teas and beverages because of their nutritional value and therapeutic properties. Currently there is only one variety of H. sabdariffa widely available for purchase known as ‘Thai red.’

### SUMMARY OF THE INVENTION

The present invention is a new variety of nutritious and edible high-biomass and seed producing hibiscus with a unique edible green calyx, increased resistance to P. japonica (Japanese beetles), and designated ‘Yao green.’

‘Yao green’ is the result of more than fifteen years of mass selection breeding in the Washington, D.C. area starting with hibiscus seeds brought from Togo in 2007. The plants were open-pollinated and seeds were harvested annually from robust, disease and pest free plants and used to propagate the next season's crop. The result is a novel, uniform, and stable local adaptation of edible H. sabdariffa that has high production value in the Mid-Atlantic region of the U.S.

The primary immediately distinguishing trait of ‘Yao green’ is its green calyx, which has remained uniform on all plants after repeated propagation from seeds, even when grown next to ‘Thai red,’ which has a red calyx. ‘Yao green’ is also a slightly larger plant in most of its physical traits, has more spacing between branches, and produces more seeds per calyx, on average, than ‘Thai Red,’ which is important for commercial growers.

Insect pests, leaf and flower yield, and mineral content of leaves of ‘Yao green’ were compared against ‘Thai red’ and five genotypes from the USDA USDA-ARS Plant Genetic Resources Conservation Unit when grown on a green roof, field row, and high tunnel. Hereafter, the varieties and genotypes will collectively be called “varieties.” ‘Yao green’ was only one of three varieties that produced flowers and it produced a moderate number of flowers. ‘Yao green’ was a top producer of leaves in all systems, was least attacked by Japanese beetles, and had higher levels of minerals than some other varieties. ‘Yao green’ was only one of two varieties that produced edible calyces when grown in these production systems in Maryland and Washington, D.C., making it an appropriate choice for a shorter growing season.

Additionally, phenolics, sugars, and amino acids of leaves from ‘Yao green’ were compared against leaves from ‘Thai red’ and the five varieties from the USDA USDA-ARS Plant Genetic Resources Conservation Unit. Whereas the composition of phenolics was similar between ‘Yao green,’ ‘Thai red,’ and other varieties of H. sabdariffa, ‘Yao green’ was ranked in the middle to highest group for total phenolics within each production system. The total amount of amino acids in ‘Yao green’ was lower than other varieties in the field row, but comparable in the other two production systems. The total amount of sugars in ‘Yao green’ was lower than other varieties in the high tunnel, but comparable in the other two production systems.

‘Yao green,’ in short, is a novel, uniform, and stable local adaptation of edible H. sabdariffa that has high production value in the Mid-Atlantic region of the U.S.

Accordingly, there is provided according to the invention, a seed of hibiscus variety designated ‘Yao green.’ There is further provided according to the invention a hibiscus plant, or a part thereof, produced by growing a ‘Yao green’ seed. There is further provided according to the invention the pollen of a ‘Yao green’ plant, and an ovule or ovules of a ‘Yao green’ hibiscus plant.

There is further provided according to the invention a hibiscus plant, or a part thereof, having all the physiological and morphological characteristics of the hibiscus variety ‘Yao green.’

There is further provided according to the invention a tissue culture of regenerable cells produced from a ‘Yao green’ hibiscus plant.

There is further provided according to the invention protoplasts or callus produced from the tissue culture of a ‘Yao green’ hibiscus variety plant.

There is further provided a tissue culture a ‘Yao green’ variety of hibiscus plant, wherein the regenerable cells of the tissue culture are produced from protoplasts or from tissue of a plant part selected from the group consisting of leaf, pollen, embryo, immature embryo, meristematic cells, immature tassels, microspores, root, root tip, anther, flower and stalk.

There is further provided according to the invention a hibiscus plant regenerated from the tissue culture of a ‘Yao green’ hibiscus variety plant, said plant having all the morphological and physiological characteristics of hibiscus variety ‘Yao green.’

The ‘Yao green’ hibiscus variety of the present invention is the subject of Plant Variety Protection Application No. 202200340, filed Apr. 29, 2022, the entirety of which is incorporated herein by reference. Seeds of the ‘Yao green’ hibiscus variety of the present invention have also been deposited with the American Type Culture Collection (“ATCC”) under Accession No.

### BRIEF DESCRIPTION OF THE DRAWINGS

The foregoing summary, as well as the following detailed description of the preferred invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a chart showing mean number (SEM) of flowers on three varieties of Hibiscus sabdariffa in three production systems. Means with different letters are significantly different (Tukey's means separation test, P<0.05).

FIG. 2 is a chart showing mean yield (SEM) in grams of Hibiscus sabdariffa leaves per plant and per harvest across seven varieties and three production systems. Means with different letters are significantly different (Tukey's means separation test, P<0.05).

FIG. 3 is a chart showing mean number (SEM) of Popillia japonica Newman (Japanese beetles) on seven varieties of Hibiscus sabdariffa in a field row in Beltsville, Md., USA. Means with different letters are significantly different (Tukey's means separation test, P<0.05).

FIG. 4 is a chart showing mean (SEM) content of minerals that differed in Hibiscus sabdariffa leaves across three production systems in parts per million (ppm). Means with different letters within each mineral are significantly different (Fisher least significant difference means separation test, P<0.05).

FIG. 5A is a chart showing mean (SEM) content of minerals that differed in Hibiscus sabdariffa leaves across four varieties measured as a percentage of the total. Means with different letters within each mineral are significantly different (Fisher least significant difference means separation test, P<0.05).

FIG. 5B is a chart showing mean (SEM) content of minerals that differed in Hibiscus sabdariffa leaves across four varieties in parts per million (ppm). Means with different letters within each mineral are significantly different (Fisher least significant difference means separation test, P<0.05).

FIG. 6 shows a typical PCA analysis of NIR spectral data from leaves of seven varieties 273388 (1), 256041 (2), 275414 (3), 267778 (4), 286316 (5), UDC green (6), and Thai red (7) grown in a field.

FIG. 7A shows site statistical analysis (mean±standard deviation) of the peak area or amounts and of total phenolics in leaves of seven Hibiscus varieties (PI 256041 (1), PI 267778 (2), PI 273388 (3), PI 275414 (4), and PI 286316 (5), Thai Red (6), and ‘Yao green’ (7) grown under field, green roof, and high tunnel conditions.

FIG. 7B shows site statistical analysis (mean±standard deviation) of the peak area or amounts and of free soluble amino acids in leaves of seven Hibiscus varieties (PI 256041 (1), PI 267778 (2), PI 273388 (3), PI 275414 (4), and PI 286316 (5), Thai Red (6), and ‘Yao green’ (7) grown under field, green roof, and high tunnel conditions.

FIG. 7C shows site statistical analysis (mean±standard deviation) of the peak area or amounts and of free soluble sugars in leaves of seven Hibiscus varieties (PI 256041 (1), PI 267778 (2), PI 273388 (3), PI 275414 (4), and PI 286316 (5), Thai Red (6), and ‘Yao green’ (7) grown under field, green roof, and high tunnel conditions.

### DETAILED DESCRIPTION OF THE INVENTION

Table 1 provides various details on the physical characteristics of ‘Yao green’ and serves as a comparison with ‘Thai red.’ These measurements were obtained by growing the two varieties simultaneously in three production systems: a green roof (Washington, D.C.), field row (Beltsville, Md.), and a high tunnel (Beltsville, Md.), as described in more detail below.

TABLE 1
Description of physical characteristics of
‘Yao green’ and ‘Thai red.’

| Physical characteristic (averages | | |
|---|---|---|
| presented) | ‘Yao green’ | ‘Thai red’ |
| Calyx, stem, branch, and petiole color | green | red |
| Flower color | white with | white with |
| | yellow center | red center |
| Height of main stem (cm) | 164.7 | 140.8 |
| Circumference of main stem (cm) | 11.7 | 12.4 |
| Plant width at widest point (cm) | 223.5 | 158.7 |
| Number branches | 24.2 | 23.6 |
| Length of branches that arise from base of | 131.2 | 119.6 |
| main stem (cm) | | |
| Length of leaf petioles (cm) | 6.4 | 5.7 |
| Length of leaves at longest point (cm) | 13.9 | 12.7 |
| Width of leaves at widest point (cm) | 12.0 | 13.9 |
| Number lobes on leaves | 3 | 3 |
| Length of calyx (cm) | 5.8 | 4.2 |
| Width of calyx (cm) | 2.5 | 2.3 |
| Number seeds per calyx | 32.8 | 29.0 |

‘Yao green’ has the following additional distinctive combination of morphological characteristics:

| | |
|---|---|
| Plant growth habit: | Spreading |
| Plant Height: | Medium |
| Plant density of branching: | Medium |
| Plant Current-Year Branch Color: | Greenish |
| Petiole Length: | Medium |
| Leaf blade length: | Medium |
| Leaf blade width: | Medium |
| Leaf blade ration of length/width: | Slightly elongated |
| Leaf blade shape of base: | Acute |
| Leaf blade intensity of green color: | Medium to dark |
| Leaf blade lobing: | Very deep |
| Leaf blade undulation: | Absent or very weak |
| Leaf blade incisions of margin: | Many |
| Leaf blade variegation: | Absent |
| Flower Type: | Single |
| Flower attitude of outermost petals: | Strongly ascending to |
| | moderately ascending |
| Flower arrangement of outermost petals: | Moderately overlapping |
| Flower diameter: | Medium |
| Flower eye zone: | Absent |
| Petal length: | Medium |
| Petal width: | Medium |
| Petal shape: | Slightly elongated |
| Petal main color on inner side: | White |
| Petal secondary color on inner side: | Pale yellow |
| Petal distribution of secondary color: | Throughout |
| Petal incisions: | Absent or weak |
| Petal undulations: | Very weak to weak |
| Calyx color: | Red |

In addition, leaf yield, pest resistance, and mineral content, NIR spectral data, free phenolics, free amino acid and soluble sugars for the leaves of ‘Yao green’ were compared to ‘Thai red’ and five varieties of H. sabdariffa from the United States National Plant Germplasm System (USNPGS). The varieties were cultivated in three agricultural production systems: field (F), green roof (R), and high tunnel (T). The varieties from the USNPGS were selected in consultation with the geneticist that overseas Hibiscus germplasm. These varieties were considered the most likely varieties that would reach the flowering and fruiting stage in a northern temperate climate. Initially, leaf yield, pest resistance and mineral content were examined. Next, data from NIR spectral fingerprinting of the seven varieties were investigated by multivariate analysis to identify similarities and differences. This was followed by a detailed phytochemical analysis (phenolics, amino acids, and sugars) of all varieties to identify specific compounds that contributed to similarities and differences.

Plant Materials. We used three agricultural production systems at two study sites: 1) the 1,858 m² green roof at The University of the District of Columbia's (“UDS”) Van Ness campus (Washington, D.C.) and 2) field and a high tunnel at UDC's Firebird Farm (Beltsville, Md.). On the green roof, we grew plants primarily in single or cross-shaped metal planter boxes. The planter boxes measured (1.22 m×2.97 m×0.31 m) and were filled with rooflite semi-intensive green roof media (Skyland USA, Landenberg, Pa.). Firebird Farm has loam soil, and fields were covered with biodegradable black plastic mulch (Dubois Agroinovation, Saint-Remi, Quebec, Canada), whereas rows in the high tunnel were not. The high tunnel (30.48 m×9.15 m×4.57 m) was covered with a 6 mil thick double-layered polyethylene film (Sun Master Infrared Anti-Condensate Thermal Greenhouse Film, Farmtek, Dyersville, Iowa, USA), which allowed for 88% light transmission and 52% diffused light transmission.

Hibiscus Samples. We planted seeds of the seven varieties (PI 256041, PI 267778, PI 273388, PI 275414, PI 286316, ‘Thai red’ and ‘Yao green’) in a high tunnel in individual plastic cells in a 50-cell tray containing sterile potting mix. Seedlings were watered as needed and at approximately six weeks, transferred to a field and high tunnel at Firebird Farm and the green roof. We planted four replicates of each variety in a single randomized row at Firebird Farm with 1.22 m between plants. The number of replicates (four) and the spacing (1.22 m) in the high tunnel were identical to the field. We arranged plants in four randomized complete blocks on the green roof. Two plants of each of two varieties were planted in a single planter box at 0.9 m spacing. The spacing on the green roof is the recommended spacing for Hibiscus cultivation and the maximum spacing the green roof would allow. We placed plants farther apart in the field and high tunnel because we had additional space and it allowed for easier harvesting. Each variety in a planter box was a single replicate, and the two plants of each variety within planter boxes were subsamples. Plants at both sites were watered through the same automatic drip irrigation and were not fertilized during the study. Leaves were harvested for nutrient analysis after three months.

We counted the number of flowers per replicate twice per week for the full growing season. Initially, only PI 286316 flowered and did so in high numbers, so petals were harvested upon counting flowers to avoid double-counting. Calyces formed despite petal removal (petals slip off the rest of the flower structure). When ‘Thai red’ and ‘Yao green’ hibiscus plants began to flower later in the season, these petals were not removed upon counting flowers because they fall off the plants within 3 days and were also far fewer in number, so removal was not necessary. ‘Thai red’ and ‘Yao green’ hibiscus are also known to produce edible calyces, so we took care not to disrupt calyx production. Calyces were counted and harvested for nutritional analysis, but since only ‘Thai red’ and ‘Yao green’ hibiscus plants produced calyces, the results are not included in this study.

Leaf yield was again determined by marketable and non-marketable weight from two harvests in each production system. We also conducted visual assessments twice per week and noted leaf damage, weather damage to leaves and branches, invertebrates, and presence of buds. Herbivorous insects were removed from plants, identified, and counted.

Harvested samples were freeze-dried, ground into fine powder, and then shipped to Waypoint Analytical (Leola, Pa.), which analyzed the amount of 12 minerals, including calcium (%), magnesium (%), nitrogen (%), phosphorous (%), potassium (%), sodium (%), sulfur (%), boron (ppm), copper (ppm), iron (ppm), manganese (ppm), and zinc (ppm).

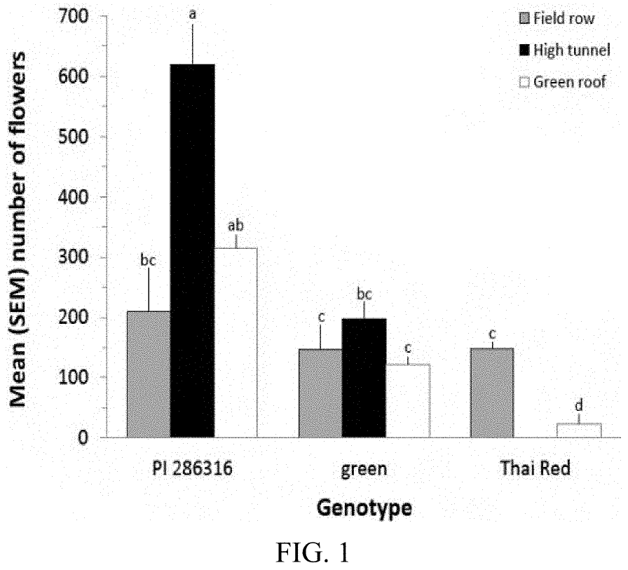

Leaf Yield and Insect Pests. PI 286316, ‘Yao green,’ and ‘Thai red’ were the only varieties that produced flowers. The number of flowers differed across production systems (x²=6.6, df=2, P=0.04), variety=13.8, df=2, P=0.001), and the variety by system interaction (x²=15.9, df=3, P=0.001). PI 286316 produced the most flowers in the high tunnel and green roof, whereas ‘Thai red’ produced the fewest flowers on the green roof (FIG. 1). All three varieties produced a relatively moderate number of flowers in the field row (FIG. 1).

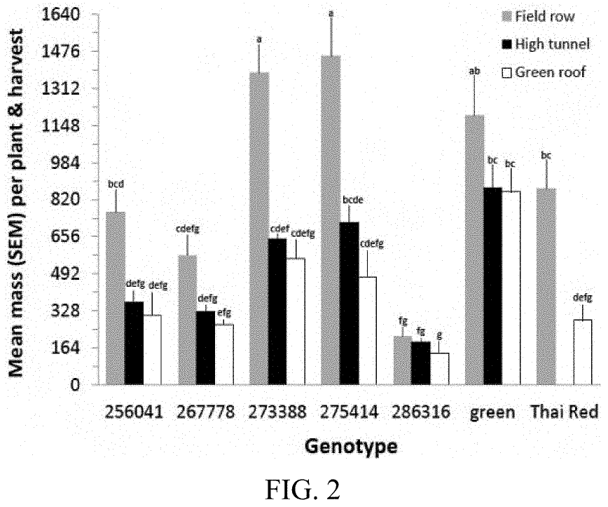

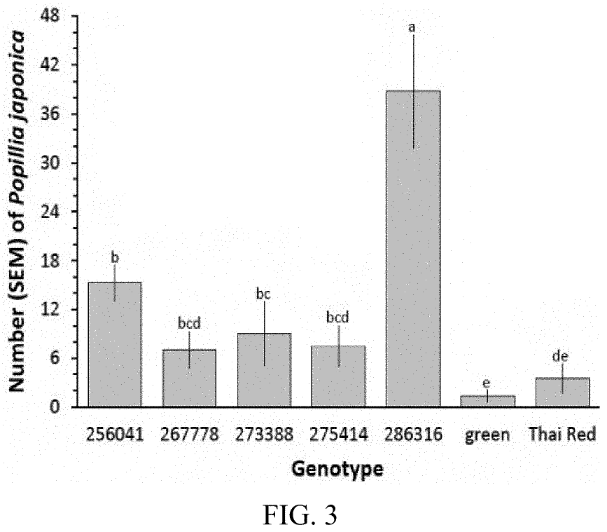

The mass of marketable leaves differed across production systems (F_{2,77}=54.6, P<0.001), variety (F_{6,77}=30.5, P<0.001), and the variety by system interaction (F_{11,77}=3.6, P<0.001). The mean yield per plant and per harvest in the field row (912±92 g) was 1.7 times higher than mean for the high tunnel (535±55 g) and 2.2 times higher than the mean for the green roof (414±51 g). Whereas varieties PI 273388, PI 275414, and ‘Yao green’ yielded the highest mass of leaves within each of the three production systems, variety PI 286316 had the lowest. (FIG. 2). ‘Thai red,’ PI 256041, and PI 267778 yielded a moderate mass of leaves in the production systems (FIG. 2). P. japonica were more than 2.5 times more abundant on variety PI 286316 in the field row than any other varieties and 11 and 29 times more abundant than on ‘Thai red’ and ‘Yao green,’ respectively, which had the lowest number of beetles (FIG. 3).

Minerals in Leaves. Harvested samples were freeze-dried, ground into fine powder, and then shipped to Waypoint Analytical (Leola, Pa.), which analyzed the amount of 12 minerals, including calcium (%), magnesium (%), nitrogen (%), phosphorous (%), potassium (%), sodium (%), sulfur (%), boron (ppm), copper (ppm), iron (ppm), manganese (ppm), and zinc (ppm). The amount of 10 of 12 minerals we analyzed varied across production systems; only the amount of sodium and boron were similar across systems (Table 2).

TABLE 2
Results of parametric and non-parametric models that tested differences
in leaf minerals among three production systems (field row, high tunnel,
green roof) and four varieties of Hibiscus sabdariffa (PI 273388,
PI 275414, ‘Yao green,’ ‘Thai red’), including the interacting
between system and variety (AKA “genotype”).

| Dependent | | Test | | |
|---|---|---|---|---|
| variable | Term | statistic | df | P |
| Calcium | System | F = 27.3 | 2 | <0.01 |
| | Genotype | F = 33.4 | 3 | <0.01 |
| | System × genotype | F = 1.0 | 4 | 0.45 |
| Magnesium | System | F = 63.8 | 2 | <0.01 |
| | Genotype | F = 7.8 | 3 | <0.01 |
| | System × genotype | F = 0.7 | 4 | 0.59 |
| Nitrogen | System | F = 49.4 | 2 | <0.01 |
| | Genotype | F = 2.4 | 3 | <0.10 |
| | System × genotype | F = 1.35 | 4 | 0.29 |
| Phosphorous | System | F = 4.0 | 2 | 0.03 |
| | Genotype | F = 1.0 | 3 | 0.42 |
| | System × genotype | F = 0.2 | 4 | 0.92 |
| Potassium | System | F = 41.4 | 2 | <0.01 |
| | Genotype | F = 5.1 | 3 | <0.01 |
| | System × genotype | F = 0.4 | 4 | 0.84 |
| Sodium | System | F = 1.8 | 2 | 0.20 |
| | Genotype | F = 3.8 | 3 | 0.03 |
| | System × genotype | F = 0.8 | 4 | 0.57 |
| Sulfur | System | F = 14.4 | 2 | <0.01 |
| | Genotype | F = 5.4 | 3 | <0.01 |
| | System × genotype | F = 0.5 | 4 | 0.74 |
| Boron | System | F = 1.5 | 2 | 0.25 |
| | Genotype | F = 7.6 | 3 | <0.01 |
| | System × genotype | F = 1.8 | 4 | 0.18 |
| Copper | System | F = 25.2 | 2 | <0.01 |
| | Genotype | F = 2.1 | 3 | 0.14 |
| | System × genotype | F = 1.2 | 4 | 0.36 |
| Iron | System | F = 34.6 | 2 | <0.01 |
| | Genotype | F = 0.5 | 3 | 0.70 |
| | System × genotype | F = 0.1 | 4 | 0.97 |
| Manganese | System | F = 181.0 | 2 | <0.01 |
| | Genotype | F = 7.3 | 3 | <0.01 |
| | System × genotype | F = 2.7 | 4 | 0.06 |
| Zinc | System | F = 17.1 | 2 | <0.01 |
| | Genotype | F = 0.0 | 3 | 0.99 |
| | System × genotype | F = 0.4 | 4 | 0.79 |

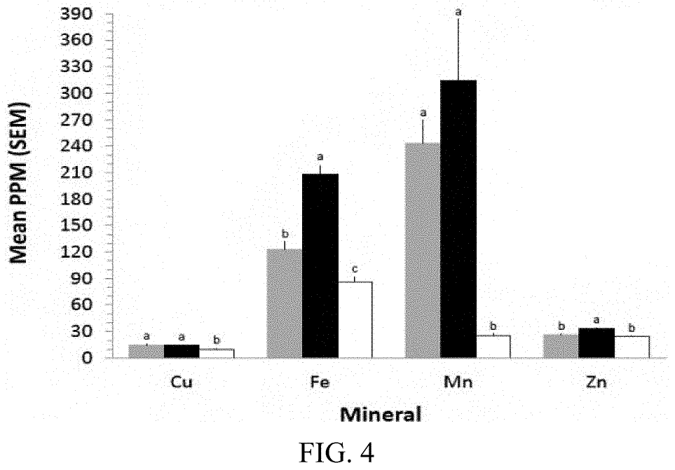

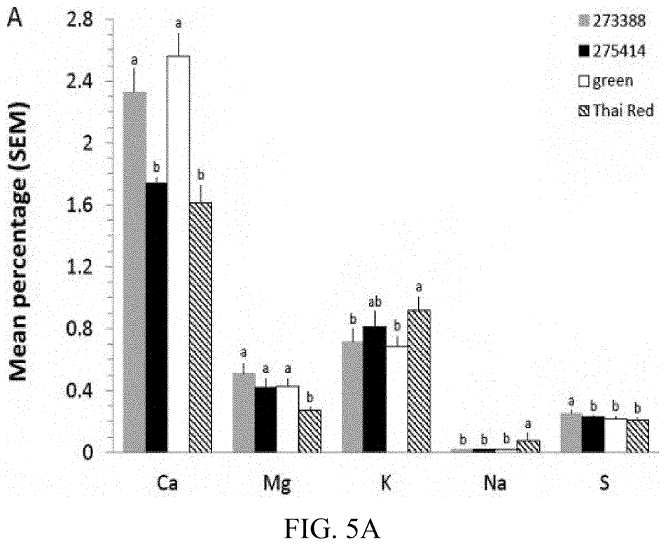

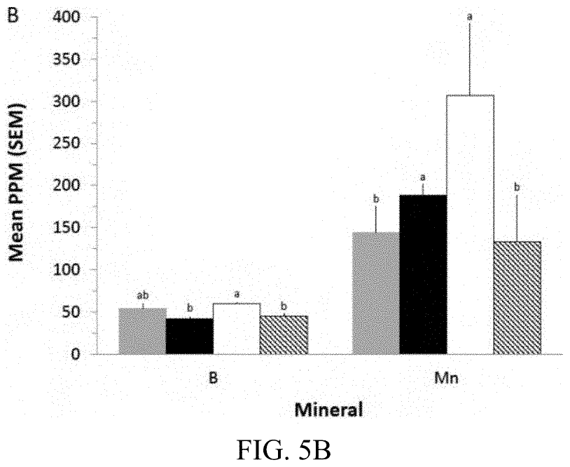

Seven of 12 minerals also varied across varieties, although nitrogen, phosphorous, copper, iron, and zinc were similar across varieties (Table 2). There were no significant interactions between production system and variety (Table 2). When analyzed by production system, hibiscus in the high tunnel had the highest amount of seven minerals, followed by the field row with six minerals, and the green roof with two minerals (FIG. 4). When analyzed by variety, PI 273388 and ‘Yao green’ each had the highest amount of four minerals (FIG. 5). PI 273388 was highest in calcium, magnesium, sulfur, and boron. ‘Yao green’ was highest in calcium, magnesium, boron, and manganese. PI 275414 had the highest amount of three minerals (magnesium, potassium, and manganese), whereas ‘Thai red’ only had the highest amount of potassium and sulfur (FIG. 5).

NIR Spectral Fingerprints. NIR spectral fingerprints of three biological replicate samples were analyzed using a Nicolet 6700 FT-IR with OMNIC software. Ground, freeze-dried leaves of each variety from three farming conditions were separately placed in a 4 mL glass vial and mixed thoroughly, and spectral fingerprints were recorded in triplicates. Diffuse reflectance spectra were collected from 10000-4000 cm⁻¹ at a resolution of 4 cm⁻¹ using an integrating sphere.

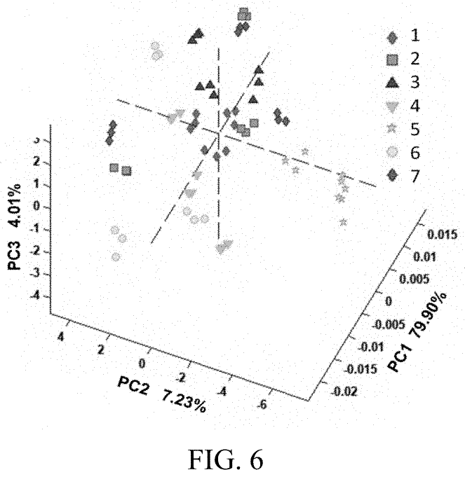

Samples of all varieties were analyzed separately within each of the three production systems to identify differences in spectral data across varieties. We presented the data from field conditions as an example of a typical PCA of NIR spectral fingerprint data of Hibiscus leaves (FIG. 6). Similar results were obtained for samples from the green roof and high tunnel.

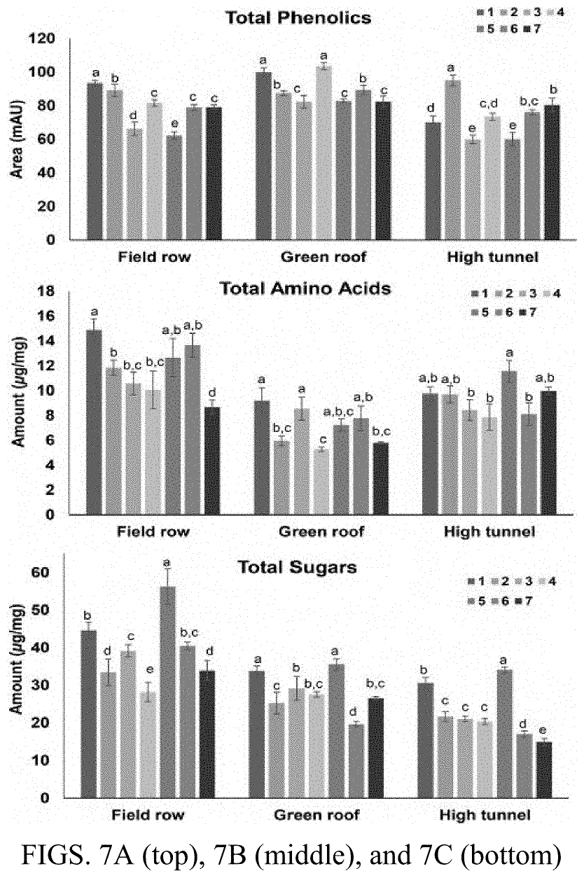

Phenolics. Screening of free phenolics by UHPLC-MS and UV spectral data, followed by comparison with previous studies, resulted in the identification of 16 compounds Comparison of total phenolics is shown in FIG. 7A.

Free Amino Acids. Edible plants provide a rich source of amino acids, and their concentration is of enormous importance in terms of nutrition. Therefore, it is necessary to explore the free amino acids in edible plants for their nutritional values. Nineteen amino acids have been reported in Hibiscus leaves after protein hydrolysis. We identified ten free amino acids: histidine, arginine, asparagine, glutamine, serine, aspartic acid, glutamic acid, glycine, GABA, and α-alanine. The highest levels (−60% more) of amino acids were in varieties grown under field conditions, whereas intermediate levels were in varieties from the high tunnel, and the lowest levels were in varieties from the green roof (FIG. 7B). Variations in the amounts of amino acids among the production systems could be due to variations in soil type and other environmental factors. The amino acid content varied by 7-50% among varieties, with higher amounts of total amino acids found in PI 256041, PI 286316, and ‘Thai red,’ whereas the lowest amounts were quantified in PI 275414 and UDC green. PI 267778 and PI 273388 had intermediate levels of free amino acids. There were three or four varieties that had the highest level of amino acids within each production system, and six of the seven varieties (except for PI 275414) had high amino acids in at least one production system (FIG. 7B).

Soluble Sugars. Previously, fructose and glucose were reported in Hibiscus samples, with glucose being the most abundant (6.5 g 100 g⁻¹ dry weight basis). We compared the variation of individual soluble sugars in seven varieties grown under three conditions. The three free sugars identified in leaves of all Hibiscus varieties were glucose, sucrose, and fructose, with sucrose being the most abundant sugar (−55%). The free total soluble sugar ranged between 8.36 to 48.3 g/mg in all seven varieties (FIG. 7C).

Presently, urban and rural farmers in the United States can only purchase ‘Thai red.’ This variety, however, was not a top yielding variety and was not as high in some minerals as other varieties. However, it, along with ‘Yao green,’ was the least attacked by P. japonica in the field row, which is the only production system where ‘Thai red’ performed as well as others. Our results indicate that local adaptation is valuable because ‘Yao green,’ which was cultivated in the Washington, D.C. area, produced a moderate number of flowers, was a top producer of leaves in all systems, was least attacked by P. japonica, and had higher levels of nutrients than most other varieties. We did not analyze the number of calyces or nutrient content of calyces as part of this project, but ‘Thai red’ and ‘Yao green’ were the only two that produced edible calyces, and they produced approximately equal numbers of calyces with similar amounts of nutrients. This information is worth considering if calyces are the primary crop.

The five varieties we tested from the USDA's germplasm repository have some notable traits, including one variety with high flower production and two with high leaf production, but the variety most production-ready for the Washington, D.C. area proved to be the locally bred, selected and adapted ‘Yao green.’

It will be appreciated by those skilled in the art that changes could be made to the preferred embodiments described above without departing from the inventive concept thereof. It is understood, therefore, that this invention is not limited to the particular embodiments disclosed, but it is intended to cover modifications within the spirit and scope of the present invention as outlined in the present disclosure and defined according to the broadest reasonable reading of the claims that follow, read in light of the present specification.

1. A seed of hibiscus variety designated ‘Yao green,’ representative seed of said variety having been deposited under ATCC Accession No: ______.

2. A hibiscus plant, or a part thereof, produced by growing the seed of claim 1.

3. Pollen of the plant of claim 2.

4. An ovule or ovules of the plant of claim 2.

5. A hibiscus plant, or a part thereof, having all the physiological and morphological characteristics of the hibiscus variety ‘Yao green,’ representative seed of said variety having been deposited under ATCC Accession No: ______.

6. A tissue culture of regenerable cells produced from the plant of claim 2.

7. Protoplasts or callus produced from the tissue culture of claim 6.

8. The tissue culture of claim 6, wherein the regenerable cells of the tissue culture are produced from protoplasts or from tissue of a plant part selected from the group consisting of leaf, pollen, embryo, immature embryo, meristematic cells, immature tassels, microspores, root, root tip, anther, flower and stalk.

9. A hibiscus plant regenerated from the tissue culture of claim 6, said plant having all the morphological and physiological characteristics of hibiscus variety ‘Yao green,’ representative seed of said variety having been deposited under ATCC Accession No: ______.
